(19) United States
(12) Patent Application Publication
(10) Pub. No.: US 2011/0300126 A1
(43) Pub. Date: Dec. 8, 2011

(54) DISPERSION AND DEBUNDLING OF CARBON NANOTUBES USING GEMINI SURFACTANT COMPOUNDS

(76) Inventor: Marianna Foldvari, Kitchener (CA)

(21) Appl. No.: 13/128,871

(22) PCT Filed: Nov. 11, 2009
(86) PCT No.: PCT/IB2009/007592

§ 371 Date: Aug. 10, 2011

Related U.S. Application Data
(60) Provisional application No. 61/113,585, filed on Nov. 11, 2008.

(51) Int. Cl. A61K 47/04; B82Y 5/00; B82Y 40/00

(52) U.S. Cl. 424/125; 977/842; 977/742; 977/906; 977/750; 977/752

(57) ABSTRACT

The current application relates to a method for solubilizing (dispersing and debundling) of carbon nanotubes using a gemini surfactant, which has head groups and a spacer linking the head groups. The dispersion of nanotubes produced by said method can be used as a delivery system for biologically active agents to an organism.

### CROSS-REFERENCE TO RELATED APPLICATIONS

This application claims the benefit of U.S. Provisional Application No. 61/113,585, filed Nov. 11, 2008, incorporated herein by reference in its entirety.

### TECHNICAL HELD

The compositions, systems, and methods relate to producing a dispersion of carbon nanotubes using gemini surfactants. The described compositions, systems, and methods are useful, e.g., for producing an environment for manipulating carbon nanotubes, and for delivering a dispersion of carbon nanotubes to an organism as a therapeutic agent.

### BACKGROUND

Carbon nanotubes (CNTs) have potential appiications in nanomedicine as biocornpatible and supportive substrates, and as pharmaceutical excipients for creating versatile drug delivery systems. Carbon nanotubes can be used as additives to improve the solubility and bioavailability of poorly soluble drugs, delivery vehicles to improve both circulatory persistence and targeting of drugs to specific cells, as carriers to improve controlled drug release, as adjuvants for vaccine delivery, for diagnostic purposes, and for drug delivery.

Carbon nanotubes have distinct structural properties that make them well-suited for these and other applications, including a high aspect ratio, ease of functional modification, and biocompatibility. However, difficulties in solubilizing carbon nanotubes represented sigMicant obstacle to their application.

### SUMMARY

In one aspect, a method for solublizing carbon nanotubes is provided. The method comprises contacting carbon nanotubes with a gemini surfactant having head groups and a spacer linking said head groups, wherein said contacting produces a dispersion of nanotubes.

In one embodiment, the gemini surfactant may be a cationic gemini surfactant.

Particular gemini surfactants may have a structure selected from:

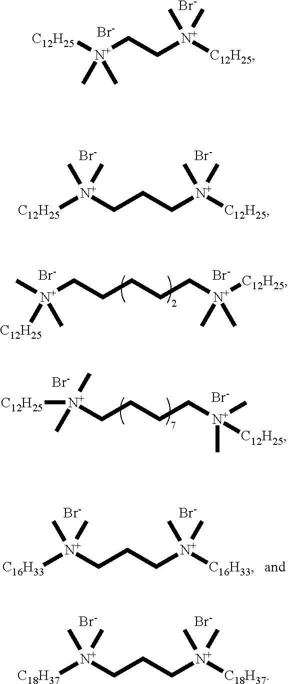

In another embodiment, the gemini surfactant is one having an m-s-m configuration, where m is the number of alkyl carbon atoms in a tail of the surfactant and s is the number of alkyl carbon atoms in a spacer. Exemplary m-s-m type surfactants are selected from the group consisting of 12-2-12, 12-3-12, 12-7-12, 12-16-12, 16-3-16, and 18-3-18. That is, in one embodiment, the gemini surfactant has 12, 16, or 18 carbon atoms in an alkyl tail portion, and 2, 3, 7, or 16 carbon atoms of an alkyl type in a spacer portion.

The extent of solubilization of the carbon nanotubes in the Gemini surfactant may be determined by optical microscopy, by Raman microscopy, by measuring the zeta potential of the dispersion, and/or by measuring particle size in the dispersion. In some cases, the zeta potential is greater than about +30 mV.

Solublizing may include dispersing and/or debundling the carbon nanotubes.

The method may further including the step of removing carbonaceous impurities from the carbon nanotubes. This step may be performed, in various embodiments, by centrifugation, by filtration, or by other methods.

The method may further including the step of removing carbon nanotube aggregates. This step may be performed, for example, by centrifugation, filtration, or other methods.

The nanotubes can be single walled, double walled or multiwalled nanotubes.

In another aspect, a dispersion of nanotubes produced by the described method is provided.

In yet another aspect, a system for dispersing nanotubes is provided, which system uses the compositions and/or methods described.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1C show a vial containing a suspension of single-walled carbon nanotube (swNT) suspensions, along with an optical micrograph (center image) and a scanning electron microscopy (SEM) micrograph (right image), in water (FIG. 1A), DMSO (FIG. 1B), and a gemini surfactant (FIG. 1C).

FIGS. 2A-2B are graphs showing the results of Raman spectroscopy using SWNT dispersions that include different surfactants, where the dispersions in FIG. 28 are SWNTs in various gemini surfactants.

FIGS. 3A-3B are graphs showing the zeta potential of SWNT in a water dispersion (FIG. 3A) and in a dispersion including an exemplary gemini surfactant (FIG. 3B).

FIGS. 3C-3D are graphs showing the results of particle size measurements of SWNT in a water dispersion (FIG. 3C) and in a dispersion including an exemplary gemini surfactant (FIG. 3D).

FIGS. 4A-4C are a table summarizing various features of exemplary surfactants tested for their ability to solubilize carbon nanotubes.

FIG. 5 is a graph showing the concentration dependence of the zeta potential of a dispersion of carbon nanotubes using an exemplary gemini surfactant.

FIG. 5A shows the effect of centrifugation to remove carbonaceous impurities and aggregates on the zeta potential of several carbon nanotube dispersions.

FIG. 6B shows the effect of centrifugation to remove carbonaceous impurities and aggregates on the particle size of several carbon nanotube dispersions.

FIGS. 7A-7H are transmission electron microscopy photomicrograps of dispersions comprised of multiwalled carbon nanotubes dispersed in a 12-3-12 gemini surfactant, where the photomicrograph on the left side shows individually dispersed nanotubes and the photomicrograph on the right side is a higher resolution image of the nanotube, where the nanotubes have diameters of 8-15 nm (FIGS. 7A-7B), 20-30 nm (FIGS. 7C-7D), 20-40 nm (FIGS. 7E-7F) and greater than 50 nm (FIGS. 7G-7H).

FIG. 8 is a graph showing the UV absorbance at 500 nm as a function of concentration, of multiwalled carbon nanotubes dispersed in three Gemini surfactants (18-3-18, diamonds; 16-3-16, squares; 12-3-12, triangles), in sodium dodecylsulphate (x), in TWEEN 80 (*) and in TWEEN 60 (circle).

### DETAILED DESCRIPTION

Gemini surfactants are a family of compounds generally characterized by having a hydrocarbon chain (referred to in the relevant art as a “tail”) connected to an ionic head group, which is connected via a spacer to another ionic head group connected to a second hydrocarbon chain (tail). The structures of gemini surfactants vary, and range from the simple m-s-m type, where m is the number of alkyl carbon atoms in the tail and s is the number of alkyl carbon atoms in the spacer, (Bombelli, C. et al., J Med. Chem., 48:5378-82 (2005); Badea, I. et al. J Gene Med., 7:1200-14 (2005); Rosenzweig, H., Bioconjug Chem., 12:258-63 (2001)) to more compiex peptide-based (Kirby, A, et al., Angew Chem int Edit, 42:1448-57 (2003)) and carbohydrate-based surfactants (Bell, P. et al., J Am Chem. Soc., 125:1551-58 (2003); Bergsma, M. et al., J Colloid Interf Sci., 243:491-95 (2001); Fielden, M. et al., Eur J. Biochem., 268:1269-79 (2001); Johnsson, M. et al., Langmuir, 19:4609-18 (2003); Johnsson, M. et al., J Chem SOC., 125:757-60 (2003); Johnsson, M, et al., J Phys Org. Chem., 17934-44 (2004); Yoshirnura, et al., Langmuir, 21:10409-15 (2005)). Some gemini surfactants form a complex with biologically active agents (e.g., nucleic acids), which complex can be transfected into a cell.

The present compositions, systems, and methods are based, in one embodiment, on the unexpected observation that gemini surfactants are effective in solubilizing (i.e., dispersing and/or unbundling) carbon nanotubes, allowing the preparation of carbon nanotube dispersions for manipuiation, modification, and delivery to an organism. Observations and results in support of the present compositions, systems, and methods are described in detail, below.

In studies conducted in support of the claimed methods and compositions, various techniques were used to determine the morphology of carbon nanotubes in different solvents and to establish a system and method for measuring and describing dispersions of carbon nanotubes. Exemplary carbon nanotubes used in the study included both single-wailed carbon nanotubes (SWNTs) and multi-walled carbon nanotubes (MWNTs). These studies will now be described with reference to the Examples and drawings.

In a first study, detailed in Example 1, SWNTs were dispersed in several exemplary solvents, water, propylene glycol (PG), dimethylsuifoxide (DMSO), ethanol, or in aqueous solutions of anionic, cationic and neutral surfactants. The dispersions were sonicated and then evaluated using zeta V)) potential, dynamic light scattering, Raman spectroscopy, optical microscopy, and scanning electron microscopy (SEM). Size and zeta potential measurements were taken within an hour after sonication, while the dispersion stability study was conducted over a nine month period.

The carbon nanotube suspensions were assigned to one of three categories: insoluble, swollen or dispersed, based on optical microscopy and SEM observations of the dispersions. An insoluble suspension was characterized by aggregation and sedimentation of the carbon nanotubes soon after sonication, where the carbon nanotubes were visible as a sedimentation at the bottom of the vial. “Swollen” suspensions were characterized by carbon nanotube aggregates visible in suspension and as a sediment at the bottom of the vial, and SEM images showing smaller aggregates or bundles of carbon nanotubes. A “dispersed” solution of carbon nanotubes was characterized by the presence of no visible aggregates in optical micrographs of the solution, and SEM images revealing exfoliated carbon nanotubes with individual, nanosized bundles.

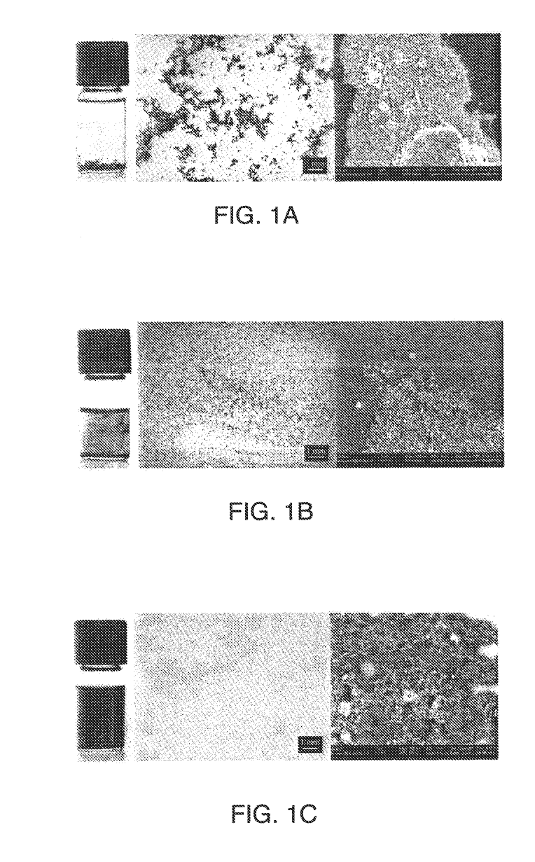

FIGS. 1A-1C show results from carbon nanotube dispersions in water, DMSO, and in a gemini 12-3-12 surfactant, respectively. As seen in FIG. 1A, carbon nanotubes in water are insoluble, with the sediment in the bottom of the vial visible and the optical (center image) and SEM (right image) images showing aggregation of the carbon nanotubes.

Swollen dispersions were obtained using propylene glycol (PEG), dimethyl sulfoxide (DMSO) and ethanol as solvents. FIG. 1B shows the results for SWNTs in DMSO. The dispersions appeared as flocculated suspensions, in which some aggregates remained in suspension and others accumulated at the bottom of the vial. SEM images revealed smaller aggregates/bundles of carbon nanotubes.

Dispersed samples were characterized by the absence of aggregates as observed by optical microscopy, while SEM micrographs show exfoliated carbon nanotubes, resulting in individul/nanosized bundles. Exfoliation (i.e., debundling) is a necessary step in the formation of carbon nanotubes dispersions, since carbon nanotubes are often provided in the form of large bundled aggregates. Dispersed suspensions had a dark even color, even when there was no visible precipitate. FIG. 1C shows the results of SWNTs in a gemini surfactant 12-3-12, which forms a dispersion.

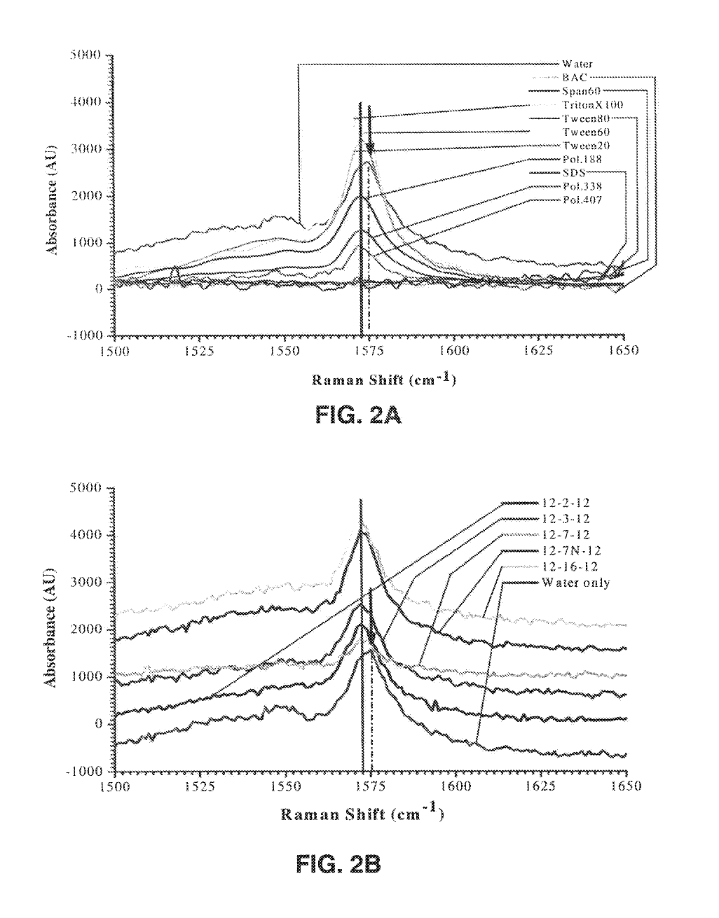

As shown in FIGS. 2A-2B, Raman spectroscopy analysis of the dispersions prepared in this study showed a shift in the G-band peak to higher wavelengths, and increased intensity, when carbon nanotube dispersions were prepared using surfactants, as compared to water alone. This shift in the G-peak, together with a G-peak intensity increase, was indicative of exfoliation (i.e., debundling) (Sinani et al., JACS, 127:8463-3472 (2005)). All gemini surfactant dispersions showed an even peak shift of ˜0.4 crn⁻¹ (1572 cm⁻¹) compared to the control dispersion in distilled deionized water (ddH₂O), which had a peak at 1576 crri⁻¹. These results were consistent with microscopy images.

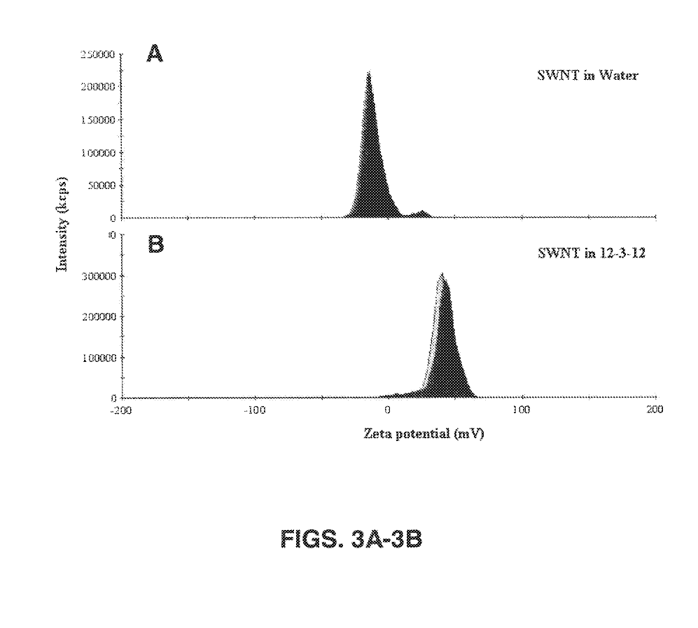

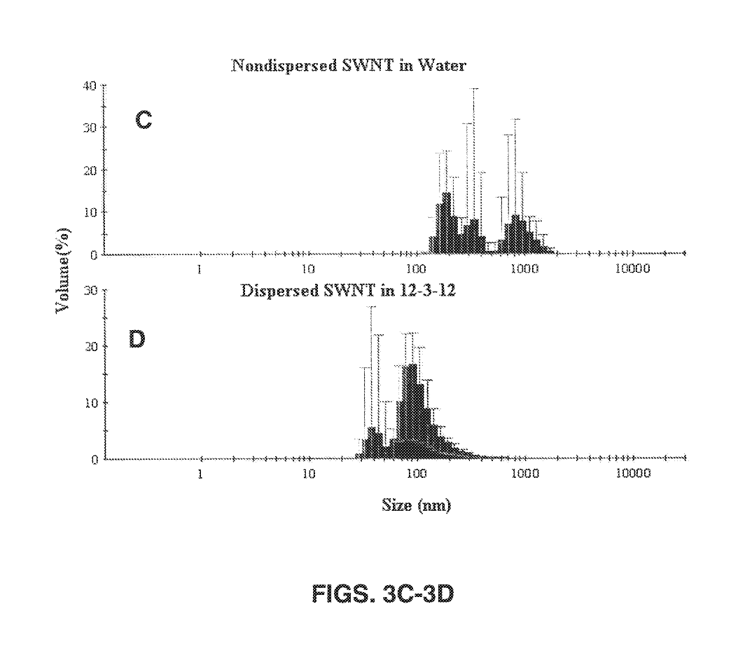

FIGS. 3A-3B snow a comparison of the stability of dispersions in water (FIG. 3A) and in an exemplary gemini surfactant (FIG. 3B) evaluated by measuring zeta potential distribution. The zeta potential was calcuiated using the Smoluchowski equation. According to the Derjaguin-Landau-VenNey-Overbeek (DLVO) theory, stable colloidal dispersions are expected to have a zeta potential expected <−35 mV and >+35 mV (Vaisman et al., Adv. Funct. Mater, 16:357-363 (2006)). Zeta potential measurements of the fully dispersed carbon nanotubes showed typical values of greater than +30 my, whiie non-dispersed samples were less than +20 mV.

FIGS. 3C-3D show a comparison of the particle size distribution of carbon nanotube dispersions in water (FIG. 3C) and in an exemplary gemini surfactant (FIG. 3D). Particle size is a hydrodynamic estimate of the anisotropic carbon nanotube dispersions. The results indicated a trend toward reduced average particle/bundle size for carbon nanotubes dispersed in an exemplary gemini surfactant. Observations made using optical microscopy were confirmed by SEM of individual carbon nanotubes. SEM images of the dispersed solution showed a significant increase in the number of dispersed carbon nanotubes with diameters <2 nm. Polydispersity in size distribution was attributed to the alignment of individual nanotubes to the polarization direction of the incident laser beam in the light source (dynamic light scattering).

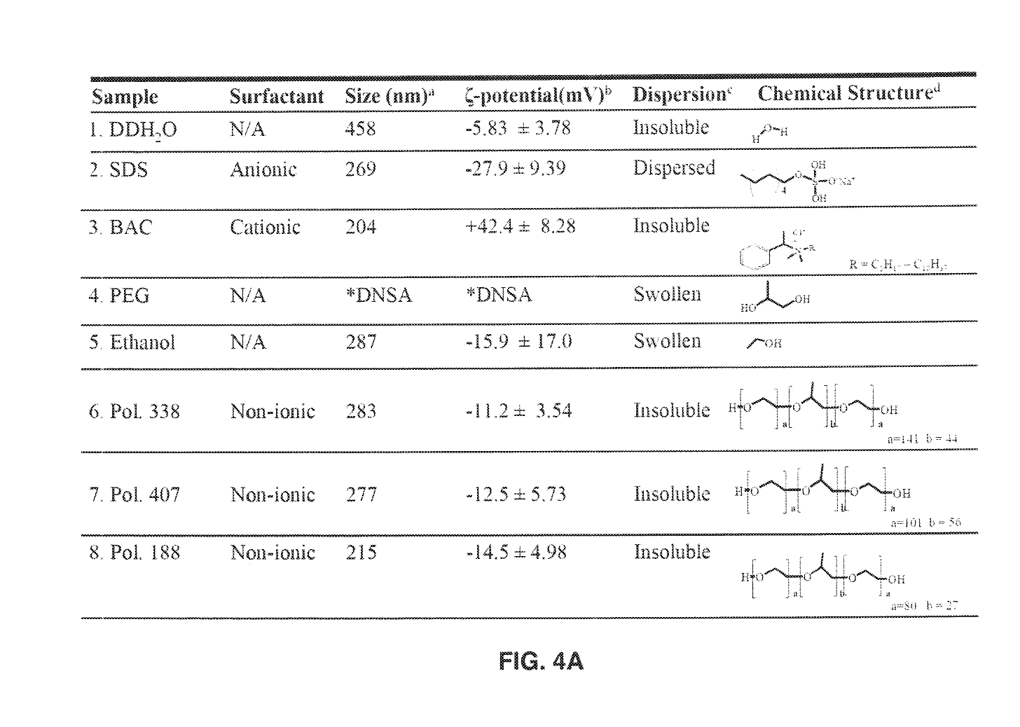

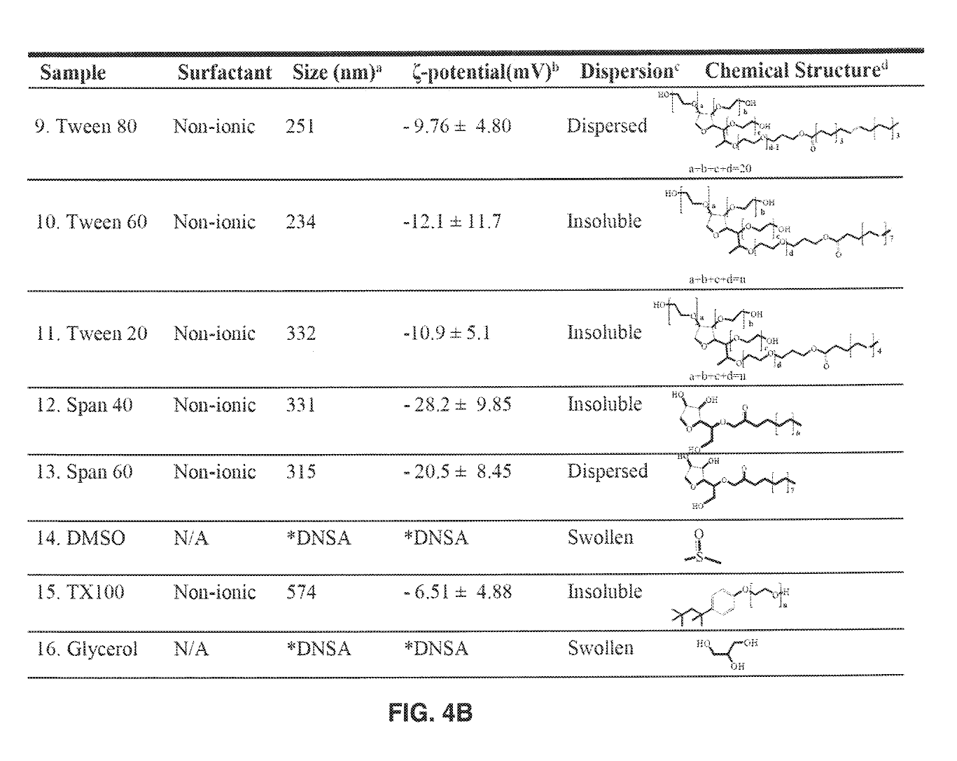

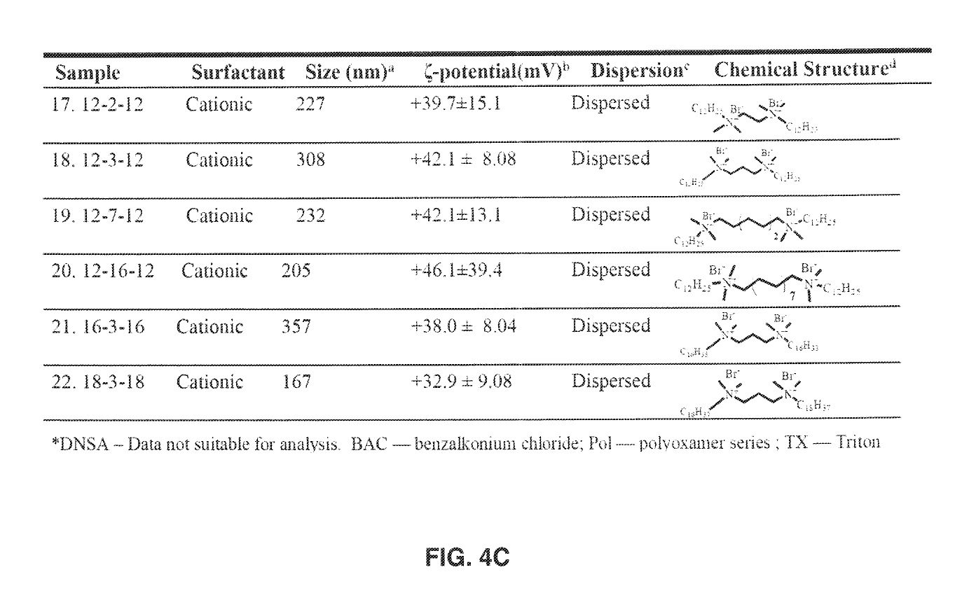

Results obtained using a variety of surfactants, including sodium dodecylsulphate (SDS), poloxamer (Poi) series (188, 338 and 407), TWEEN® series (20, 40 and 60), benzalkonium chloride (BAC), TRITON® X100 (TX-100), and a series of gemini surfactants (12-2-12, 12-3-12, 12-7-12, 12-16-12, 16-3-16, and 18-3-18), are summarized in the Table presented in FIGS. 4A-4C. Indicated in the table is the type of surfactant, observed particle size and zeta potential, the morphological characteristics of the dispersion, and the chemical structure of the surfactant.

SDS, TWEEN® 80, SPAN® 60 and several gemini surfactants showed the highest degree of solubilization/dispersion. Dispersion using ionic surfactants is believed to be mediated by interactions of the hydrophobic tail of the surfactant with the hydrophobic walls of the carbon nanotubes, and interaction of the polar head group of the surfactant with the polar solvent. These interactions are reflected by the zeta potential. Dispersion using non-ionic surfactants is mainly believed to be mediated primarily by hydrophobic tail. For non-ionic surfactants, the length of the hydrophobic tail (HT), rather than zeta potential, determines the dispersivity, i.e., dispersion obtained when HT≧10.

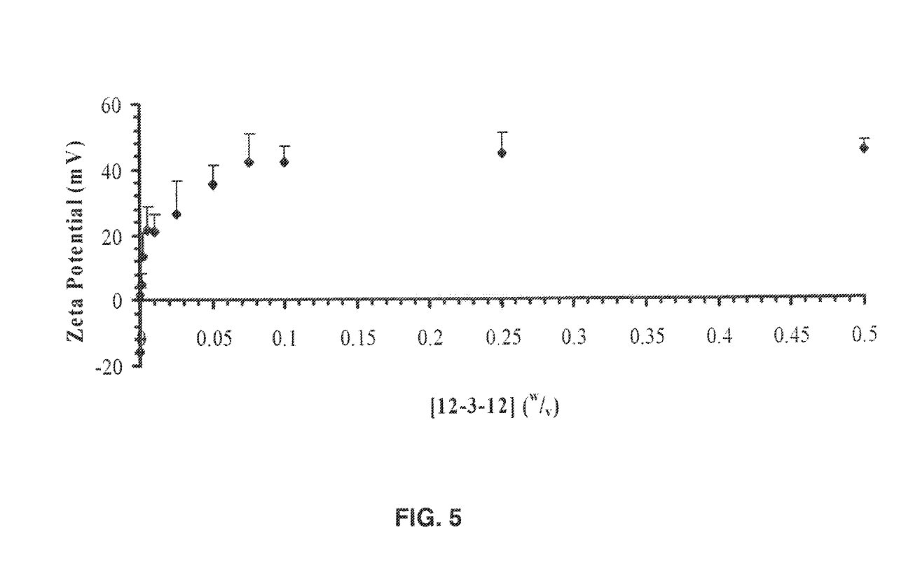

To better understand the optimal concentrations of gemini surfactants for use in dispersing carbon nanotubes, a zeta potential titration was performed using an exemplary gemini surfactant, i.e., 12-3-12. In this study, the concentration of carbon nanotubes was held at 0.1 mg/mL, while various concentration of the gemini surfactant were used. The data are shown in FIG. 5, and show an optimal concentration of gemini surfactant is in excess of 0.075 w/v, which results in a zeta potential of ≧35 mV. Accordingly, in one embodiment, a gemini surfactant in a concentration range of between 0.07-0.3 w/v, preferably 0.06-0.3 w/v in a composition comprising carbon nanotubes is provided. In a preferred embodiment, a gemini surfactant in a concentration greater than about 0.08 w/v, 0.09 w/v or 0.1 w/v is provided.

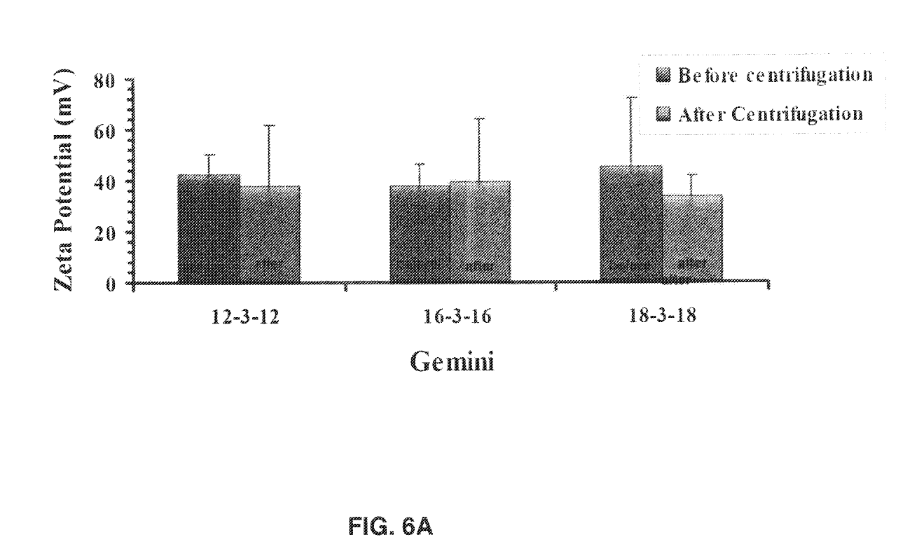

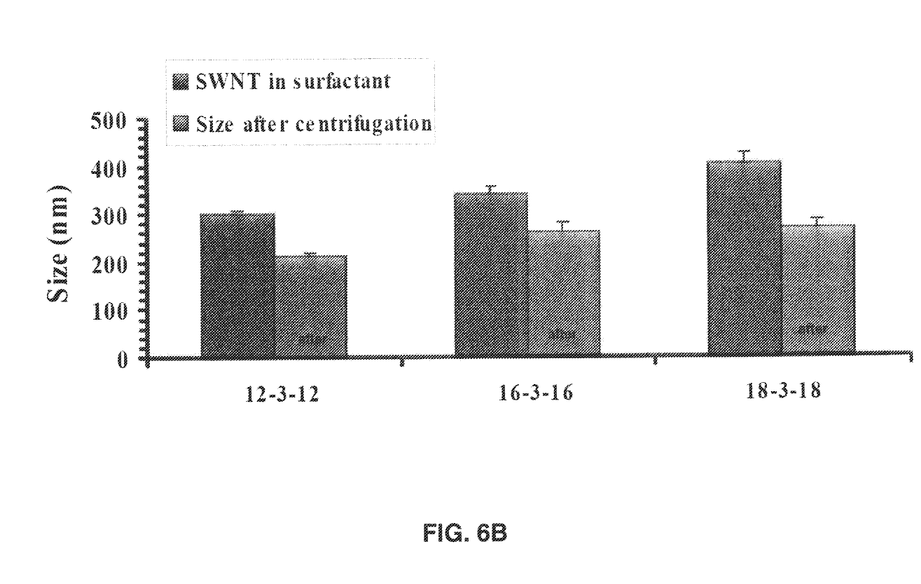

As shown in FIGS. 6A-6B, the presence of carbonaceous impurities, including larger non-dispersed bundles of carbon nanotubes can affect the dispersion of carbon nanotubes. As shown in FIG. 6A, subjecting a carbon nanotube preparation to centrifugation (e.g., 5,000×g) changed the zeta potential of the resulting gemini surfactant dispersion, although the effect on dispersion appears to depend on the particular surfactant. In contrast, as shown in FIG. 6B, subjecting a carbon nanotube preparation to centrifugation (e.g., 5,000×g) reduced the particle size obtained using each of three different gemini surfactants. Note that the gemini surfactants used in FIGS. 6A-6B included equal length spacers with different head groups.

In another study, detailed in Example 2, dispersions of multiwalled carbon nanotubes (MWNTs) were prepared. Four MWNTs were commercially obtained, and dispersions were prepared using various gemini surfactants 12-3-12, 16-3-16, 12-2-12, 12-7-12, 12-7NH-12 and 12-16-12. For comparison, dispersions of the MWNTs were also prepared with water, SDS, polyvinylpyrrolidon and DMSO. The dispersions were visually inspected to observe for sedimentation, and were characterized by transmission electron microscopy (TEM) and UV spectroscopy.

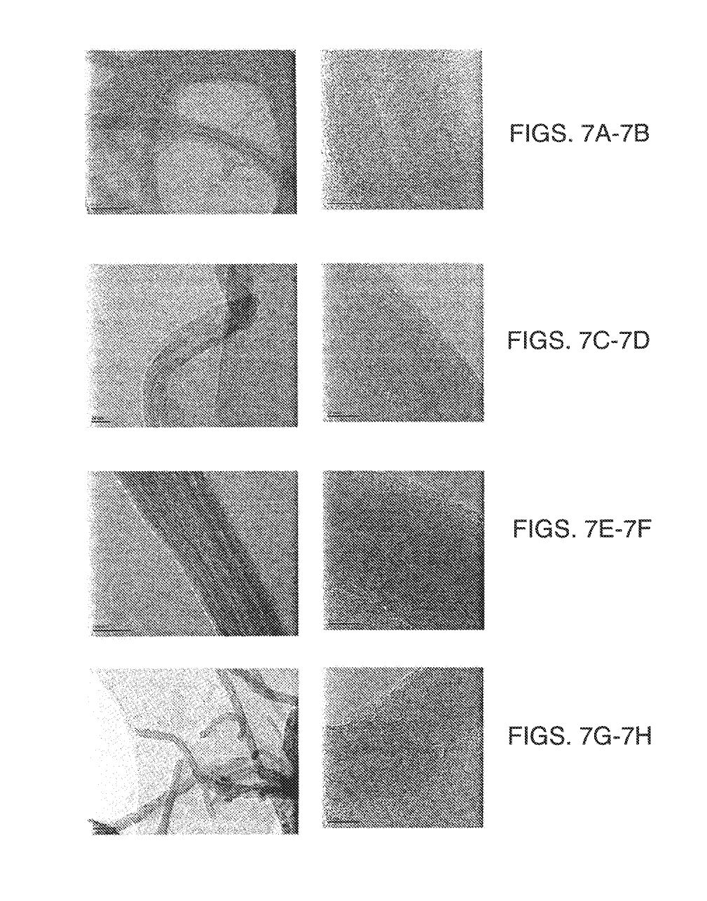

The TEM photomicrographs are shown in FIGS. 7A-7H for dispersions of the four MWNTs in a 12-3-12 gemini surfactant. The photomicrograph on the left side of each pair of images shows individually dispersed nanotubes and the photomicrograph on the right side of each pair is a higher resolution image of the nanotube. The MWNTs in the study had diameters of between 8-15 nm (FIGS. 7A-7B), between 20-30 nm (FIGS. 7C-7D), between 20-40 nm (FIGS. 7E-7F) and greater than 50 nm (FIGS. 7G-7H). It is seen that the gemini surfactant was effective to disperse the MWNTs with no aggregation of the MWNTs observed.

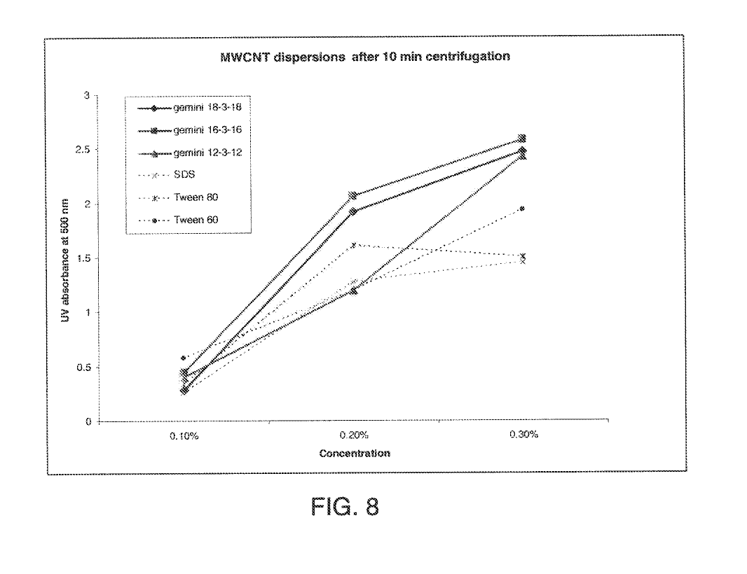

FIG. 8 is a graph showing the UV absorbance at 500 nm as a function of concentration, multiwalled carbon nanotubes dispersed in three gemini surfactants (18-3-18, diamonds; 16-3-16, squares; 12-3-12, triangles), in sodium dodecylsulphate (x), in TWEEN 80 (*) and in TWEEN 60 (circle). The data shows that gemini surfactants are particularly effective in dispersing MWNT compared to SDS, TWEEN® 80 or TWEEN® 60. Gemini surfactants with alkyl chain lengths in the tail portion of C16 and C18 achieved particularly remarkable dispersion, as evidenced by the clarity of these dispersions, compared to the C12 gemini surfactant.

Accordingly, and in one aspect, compositions are provided for dispersing, i.e., maintaining in solution or suspension without aggregation, carbon nanotubes. Such compositions may also exfoliate, i.e., debundle, carbon nanotubes that are in the form of an aggregate. The compositions may include one or more gemini surfactants, optionally with one or more additional non-gemini surfactants. In some cases, the composition includes one or more gemini surfactants, in the absence of other surfactants. In a related aspect, systems are provided for dispersing carbon nanotubes, the system including at least one gemini surfactant.

In another aspect, methods for dispersing carbon nanotubes are provided. The methods may also exfoliate, i.e., debundle, carbon nanotubes. The methods include forming an admixture of one or more gemini surfactants with carbon nanotubes. The method may optionally include the use of additional non-gemini surfactants, or may include only a gemini surfactant while excluding other surfactants.

The methods may include a step for removing carbonaceous impurities and/or carbon tubule aggregates, e.g., to improve the uniformity and consistency of the resulting carbon tubule dispersions. Exemplary steps for removing carbonaceous impurities and/or carbon tubule aggregates include but are not limited to centrifugation, and filtration.

Exemplary carbon nanotubes include but are not limited to single-walled carbon nanotubes (SWNTs); however, other types of carbon nanotubes (double walled and multi-walled), or other carbon nanostructures, can be used with the present compositions, systems, and methods.

Gemini surfactants for use as described have a hydrocarbon chain (i.e., tail) connected to an ionic head group, which is connected via a spacer to another ionic head group connected to a long hydrocarbon chain (tail). In one embodiment, the hydrocarbon tail has between about 8-24 carbon atoms, preferably between about 8-20, 8-18, 10-24, 10-20, 10-18, 12-20 or 12-18 carbon atoms, preferably alkyl carbon atoms. In one embodiment, the number of carbon atoms in the spacer moiety is 2, 3, 4, 5, 6, 7, 8, 9, 10, 11, or 12, or is between 2-5, 2-7, 3-7. In another embodiment, the two hydrocarbon tails of the gemini surfactant are of even length (a ‘symmetric’ surfactant) or are of different lengths (an “asymmetric’ surfactant). As noted above, the structures of gemini surfactants range from the m-s-m type, where m is the number of alkyl carbon atoms in the tail and s is the number of alkyl carbon atoms in the spacer, to peptide-based gemini surfactants and carbohydrate-based surfactants.

Particular gemini surfactants for use as described have the following structures, which are also referred to as 12-2-12, 12-3,12, 12-7-12, 12-16-12, 16-3-16, and 18-3-18, respectively:

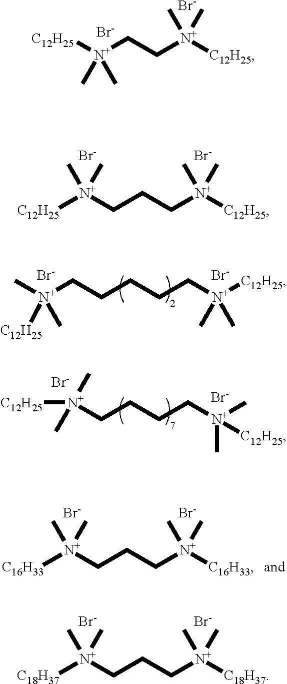

Additional gemini surfactants are those with spacer substitutions, including N-substitutions (such as the 12-7NH-12 gemini surfactant used in the study of Example 2), such as azo or imide substitution, or O-substitutions, such as hydroxyl, ether, carboxyl, or ether substitutions. That is, in one embodiment, the gemini surfactant has a spacer moiety that is modified at one or more carbon atoms with a nitrogen or an oxygen. Further additional gemini surfactants are asymmetric gemini surfactants in which one hydrocarbon tail is different from the other. Particular asymmetric gemini surfactants include a pyrene moiety. Although bromide salts are indicated, the particular counter-ion used in not critical. Additional gemini surfactants are described in WO05/039642, which is incorporated by reference herein.

### Formulations, Dosages, and Treatment

In another aspect, compositions and delivery systems comprising the carbon nanotubes dispersed in a gemini surfactant are provided. An example of a delivery system comprising multi-walled carbon nanotubes, a gemini surfactant, plasmid DNA as the therape agent, and other excipients is set forth in Example 3. The delivery system of Example is preferably administered topically, for local or systemic administration of the plasmid DNA. A skilled artisan will appreciate that delivery systems can be prepared for other routes of administration, including injection.

The carbon nanotubes may be subject to chemical modification of the surface, in some embodiments. In preparing the compositions and delivery systems, modification of the surface of the nanotubes can enhance their admixture with therapeutic agents.

Exemplary beneficial agents for use in the compositions and delivery systems include but are not limited to nucleic acids, proteins, small molecule drugs, and other therapeutic compounds. The therapeutic agent and the carbon nanotubes are formulated into, for example, creams, lotions, pastes, ointments, foams, gels and liquids, coated substrates, and transdermal patches, all of which may include suitable non-toxic, pharmaceutically acceptable carriers, diluents and excipients as are well known in the art (see for example, Merck Index, Merck & Co., Rahway, N.J.; and Gilman et al., (Edo) (1996) Goodman and Gilman's: The Pharmacological Bases of Therapeutics, 10^{th} Ed., McGraw-Hill). In a preferred embodiment, carriers, diluents, excipients or supplements are selected that are biocompatible, pharmaceutically acceptable, and suitable for administration to the skin or mucosal membrane of a subject. In another embodiment, a topical formulation comprising carbon nanotubes, a therapeutic agent, an acylated amino acid and optionally lipid vesicles is prepared. Acylated amino acids are described, for example, in PCT/CA2000/001323, published as WO01/035998, which is incorporated by reference herein. All agents are preferably non-toxic and physiologically acceptable for the intended purpose, and preferably do not substantially interfere with the activity of the biologically active agent.

The dosage of the delivery system depends upon many factors that are well known to those skilled in the art, for example, the particular form of the biologically active agent within the delivery system, the condition being treated, the age, weight, and clinical condition of the recipient animal/patient, and the experience and judgment of the clinician or practitioner administering the therapy. A therapeutically effective amount provides either subjective relief of symptoms or an objectively identifiable improvement as noted by the clinician or other qualified observer. The dosing range varies with the biologically active agent within the delivery system used, its form, and the potency of the particular agent. For standard dosages of conventional pharmacological agents, see for example, the U.S. Pharmacopeia National Formulary (2003), U.S. Pharmacopeial Convention, Inc., Rockville, Md.

Further embodiments of the compositions, systems, and methods will be apparent to the skilled artisan upon reading the disclosure. The following examples are intended to illustrate the compositions, systems, and methods but are in no way intended to be limiting.

### EXAMPLES

The following example is provided to further illustrate the compositions, systems, and methods.

### Example 1

### Formulation of Single-Walled Nanotubes

Single-wall carbon nanotubes (SWNTs) were obtained from Carbon Solutions Inc. (P/N AP-155, produced by electric arc discharge). The SWNTs were dispersed at a concentration of 0.1 mg/mL in water, propylene glycol (PG), dirhethylsultoxide (DMSO), and ethanol, or as 0.1% w/v aqueous solutions of anionic, cationic and neutral surfactants at a SWNT concentration of 0.1 mg/mL. The dispersions were sonicated for 12 hours.

The stability of the SWNT dispersions were evaluated by zeta (ζ) potential measurements (Malvern's Nano ZS). The dispersion of SWNTs in solution was analyzed by dynamic light scattering, Raman spectroscopy, optical microscopy, and scanning electron microscopy (SEM). SEM samples were prepared by transferring 5 μL of dispersed SWNTs onto pre-heated (˜150° C.) silicon substrates.

Size and zeta potential measurements were taken within an hour after sonication, whiles the dispersion stability study was conducted over a nine month period. Results obtained using these methods are shown in the accompanying FIGS. 1-6.

### Example 2

### Compositions Comprising Multi-Walled Nanotubes and Gemini Surfactants

The following multiwall carbon tubes (MWNTs) were commercially obtained (Cheaptubes.com): MWCNT15—outer diameter: 8-15 nm, length: 50 μm, purity>95%; MWCNT30—outer diameter: 20-30 nm, length: 50 μm, purity>95%; MWCNT40—20-40 nm, length: 50 μm, purity>90%; and MWCNT50—outer diameter: >50 nm, length: 50 μm, purity>90%.

Two methods for dispersion of the MWNTs were used. In Method 1, the carbon nanotubes were pre-weighed into glass vials. Gemini surfactant solutions (0.1% w/w) were added to obtain 1 mg/100 mL dispersions. The dispersions were sonicated using a Misonix cuphorn sonicator for 15 minutes, followed by bath sonication (VWR sonicator) for 5 hours. In Method 2, the carbon nanotube dispersions were prepared using the NanoDeBee high shear homogenizer (BEE International Inc) for 3 minutes on continuous cycle at temperatures up to 80° C.

The MWNT dispersions were centrifuged at 10,000 g for 5 minutes. The nanotubes the supernatant were recovered and characterized by transmission electron microscopy (TEM) and UV spectroscopy.

The following Gemini surfactants were used: 12-3-12, 16-3-16, 12-2-12, 12-7-12, 12-7NH-12 and 12-16-12. For comparison, dispersions were prepared with water, SDS, polyvinylpyrrolidon and dimethyl sulfoxide (DMSO).

The dispersions in each vial were visually inspected as a function of time. In addition, the carbon nanotube dispersions were characterized using TEM, by placing an aliquot of each dispersion on 300 mesh holey copper grids and viewing in a Jeol 2010F 200 kV FEG TEM/STEM. The concentration of the nanotubes in each dispersion was measured using UV spectroscopy, where the UV absorbance of centrifuged nanotube dispersions were measured using a Spectramax M5 multi-detection microplate reader (Molecular Devices).

Visual inspection of the dispersions revealed that gemini surfactants dispersed both SWNT and MWNT and resulted in uniform black solutions without sedimentation for at least one week. The TEM results, seen in FIGS. 7A-7H, showed the presence of individuaily dispersed nanotubes. The UV absorbance results are shown in FIG. 8, and show that gemini surfactants, at 0.2 and 0.3% w/v concentration, are particularly effective in dispersing MWNT compared to SDS, TWEEN 80 or TWEEN 60. The ionger alkyl chain, C16 and C18, gemini surfactant show higher dispersive power compared to shorter chain (C12) gemini surfactant.

### Example 3

### Delivery Systems Comprising Nanotubes and Gemini Surfactants and a Biological Agent

A topical formulation with the following composition was developed. A dispersion of 1 mg/100 mL multiwalled carbon nanotubues (1 mg, MWNT) in a 0.1% gemini surfactant (12-3-12) solution (100 mL) was prepared. The components were dispersed together by sonication using a Misonix cuphorn sonicator for 15 minutes, followed by bath sonication (VMR sonicator) for 5 hours.

Next, a complex of the carbon nanotubes with a nucleic acid was formed, 1.8 mL of the carbon nanotube dispersion was mixed with 1.8 mL of plasmid DNA (pDNA, 1.7 mg/mL stock solution). The pDNA and MWNT dispersion were briefly vortexted.

Next lipid nano-vesicles comprised of the following components were prepared: phospholipon 100H (10% w/w), propylene glycol (10% w/w), phospholipid EFA (4% w/w); palmitoyl-lauroyl lysine [N (alpha)-paimitoyl-N-(epsilon) lauroyl L-lysine methyl ester (PDM 17; 0.1% w/w), and dH₂O (ds to 100%). The first four excipients were heated in a glass vial on a water bath at 70-80° C. The fifth ingredient (water) was added to the lipid mixture at 55° C.; and the mixture was vortexed. The vesicles were processed through a NanoDeBee high shear homogenizer (BEE International Inc) for 3 individual passes.

Next, a MWCT-DNA-lipid complexes were prepared by combining 2.4 mL of the lipid nano-vesicles with 3.6 mL of the MWNT-DNA complex.

The preparation is applied topically to a subject, for topical delivery of the nucleic acid.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for solublizing carbon nanotubes, comprising:
contacting carbon nanotubes with a gemini surfactant having head groups and a spacer linking said head groups, wherein said contacting produces a dispersion of nanotubes.

2. The method of claim 1, wherein the gemini surfactant is a cationic gemini surfactant.

3. The method according to claim 1, wherein the gemini surfactant has a structure selected from:

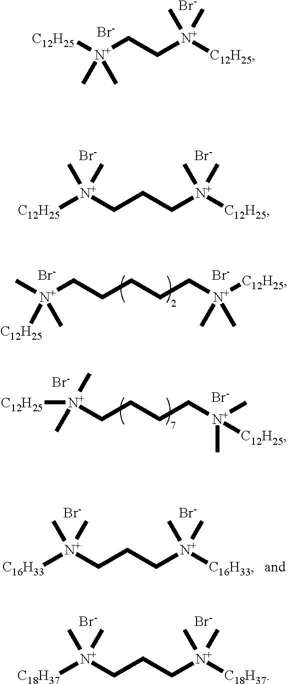

4. The method of claim 1, wherein the gemini surfactant is one having an m-s-m configuration, where m is the number of carbon atoms in a hydrocarbon tail and s in the number of carbon atoms in the spacer.

5. The method of claim 4, wherein m is 12, 16 or 18 and s is 2, 3, 7, or 16.

6. The method of claim 4, wherein the surfactant has m-s-m values selected from the group consisting of 12-2-12, 12-3-12, 12-7-12, 12-16-12, 16-3-16, and 18-3-18.

7. The method of claim 1, wherein the gemini surfactant is a gemini surfactant with an N-substitution or an O-substitution on the spacer.

8. The method according to claim 1, wherein the carbon nanotubes are single walled carbon nanotubes, double walled carbon nanotubes, or multi-walled carbon nanotubes.

9. The method of claim 1, wherein the solublizing includes dispersing and debundling the carbon nanotubes.

10. The method of claim 1, further including the step of removing carbonaceous impurities from the carbon nanotubes by centrifugation.

11. The method of claim 1, further including the step of removing carbon tube aggregates by centrifugation.

12. A dispersion of nanotubes produced by the method of claim 1.

13. A delivery system for delivery of a biologically active agent to a subject, comprising a dispersion according to claim 12.
